(19) United States
(12) Patent Application Publication
(10) Pub. No.: US 2010/0179371 A1
(43) Pub. Date: Jul. 15, 2010

(54) METHOD FOR HYDROMAGNETOTHERAPY (VARIANTS) AND DEVICE FOR REALIZING THE METHOD

(75) Inventors: Sergey Vladimirovich Pletnev, Minsk (BY); Alexander Nikolaevich Razumov, Moscow (RU)

Correspondence Address: BROOKS KUSHMAN P.C., 1000 TOWN CENTER, TWENTY-SECOND FLOOR, SOUTHFIELD, MI, 48075, US

(73) Assignee: Sergey Vladimirovich Pletnev, Minsk (BY)

(21) Appl. No.: 12/295,012

(22) PCT Filed: Mar. 28, 2007
(86) PCT No.: PCT/IB07/51088

§ 371 Date: Sep. 29, 2008

(30) Foreign Application Priority Data
Mar. 28, 2006 (EA) 200600843

(51) Int. Cl. A61N 2/00; A61H 1/00

(52) U.S. Cl. 600/9; 601/47

(57) ABSTRACT

This invention relates to the field of medicine, in particular, to the method and device for magnetotherapy and/or prophylaxis of human diseases. The therapeutic effect is achieved by simultaneously combining acoustic and vibration action in the presence of a fluid medium. The effect-producing harmonic exposure is used as an acoustic exposure. Medical solutions are used as a fluid medium. The magnetic field amplitude is modulated by an acoustic signal.

This invention relates to the field of medicine, in particular, to the therapy of various diseases and anomalies both in the human and other living organisms. The invention may be also used for treating and prophylaxis of various diseases and disorders.

Methods and apparatuses for using hydrotherapy, inclusive of in combination with other therapeutic means are known in the art. The method and device for treating diseases by combining thermohydrotherapy and phytoaeroionization are known from RU 99117644, A61H 33/06, A61H 23/00. This method combines thermohydrotherapy and phytoaeroionization, aerosolic therapy and irradiation of an aerosol flow with laser light. This method and device involve preheating of a patient with steam and/or hot air before the aerosolic therapy procedure followed by a hot shower, and the phytoaeroionization process is conducted at a temperature of 15-20° C. accompanied by a vibratory massage. The device comprises an aerosol source, an ultrasonic generator and is characterized in that the aerosol source is fixed to a hollow body in a form of a bath, which is provided with a mesh-like bed with a vibrator, a heat and steam convector is secured in the lower part of the body, the body is provided with a line for supplying water in the form of a Charcot's douche and also with an opening to which a cooling and heating convector is connected.

The above methods, however, are low-efficient because they do not produce any direct effect on the biochemical and metabolic processes in cells, specifically in internal parts of the human body and muscle tissues, and, hence, they may be used mainly as preventive methods.

A device for integrated water or water solution treatment intended for hydrotherapy is known in the art. The device comprises a body provided with water inlet and discharge manifolds. The water inlet manifold is coaxially connected with a transparent tube provided with a light-reflecting coating and disposed within toroidal magnets. A laser waveguide is mounted coaxially opposite the tube end and a heating element enclosed in a casing and coupled to a relay disposed outside the body is mounted within the body. The device provides an integrated water treatment according to the set algorithm.

This device, however, produces effect only on water, while internal parts of the body and tissues remain unaffected.

It is preferable to select as the closest prior art the magnetotherapy method involving disposal of an inductor close to an organ to be treated and treatment of this organ with a pulsed magnetic field having specific frequency and amplitude of the magnetic field pulses for a specific period of time. According to this method, a body of the patient and/or the organ to be treated are exposed to an acoustic action simultaneously with the magnetotherapy (EA No. 000495, A61N 2/02).

According to this method, the organ to be treated is exposed by the magnetic field pulse packets. The pulse packet-repetition frequency may be matched with one of biological rhythms (for example, cardiac rate, alpha rate, etc.) usually within the range of from 1 to 10 Hz. The pulse-repetition frequency in a packet is usually selected at about 100 Hz and higher. This frequency may be also matched with the resonance frequency of the magnetic permeability response of organ tissues being treated. Usually, the fields with induction of 1-50 mTl are used. In the method provided, it is preferable to use fields with induction of 1-10 mTl as not causing physiological or reflex action, but having only a stimulatory effect.

According to the disclosed invention, a simultaneous application of two means, i.e. pulsed magnetic and acoustic, with the activated biochemical processes in the organ tissues being treated and synchronizing acoustic exposure, drastically increases efficiency of therapy and allows treating some kinds of diseases, which are untreatable when each type of the actions was used separately. In this case, both the whole body may be exposed to the acoustic exposure through hearing and the organ to be treated may be exposed to it directly. In addition, an effective value of the magnetic field may be substantially reduced, thereby also reducing the probability of side effects related to overdosing and conserving energy consumption during the therapy course. In this case, if the whole body is exposed to the acoustic exposure, i.e. through organs of hearing, a favorable effect on the central nervous system produces an additional positive effect and reduces the time required for rehabilitation of the organ functioning.

To efficiently treat some diseases and injuries, however, a combination of the magnetic and acoustic exposures appears to be insufficient, hence, other means such as hydrotherapy, massage, etc. need to be used to supplement them.

The aim of the preseet invention is to provide a method promoting the improved therapeutic effect in treating various diseases without using a substantial quantity of medicaments and resorting to surgical operation. The aim of the invention is also to provide a specific device for realization of this method.

According to this invention, the hydrotherapy and magnetotherapy methods are used simultaneously and involve disposing an inductor close to a human body, preferably in proximity to the organ to be treated, exposing this organ to the pulsed magnetic field with specific magnetic field pulse frequency and magnitude for a specific period of time.

It is preferable that the patient's body and/or organ to be treated were exposed to an acoustic harmonic exposure simultaneously with the hydro and magnetotherapy. The method is characterized in that a fluid medium is disposed between the body and the inductor, and the magnetotherapy is performed in the presence of the fluid medium.

It has been found that such a combination substantially enhances the effects of different methods. It should be noted that the fluid medium, its pressure and thermal action in the process of the magnetotherapy is useful for cleaning cell membranes, dissolving and excreting decomposition products and poisons from the intercellular spaces through the skin.

Further development of the invention consists in using a harmonic sounding, in particular, musical sounding, as an acoustic exposure having a stimulating effect on the functioning of the organ being treated. In this case, the fluid medium provides a more efficient transmission of the acoustic exposure.

In accordance with the further development, the amplitude of the magnetic field pulses is modulated by an acoustic signal. The pulse packet-repetition frequency according to the further development is matched with the musical sounding rhythm, while the pulse-repetition frequency in the packet is matched with the harmonic acoustical spectrum.

Further, the organ being treated is exposed to vibration simultaneously with the magnetic and acoustic exposure.

The vibration action frequency and/or pulse packet repetition frequency are matched with the harmonic components of the acoustic exposure.

Medical solutions having stimulating and/or curing affect on the functioning of the organ being treated may be used as a fluid medium.

It is most efficient to dispose the inductors along the body and close to the organs being treated in the disclosed methods.

According to one of the embodiments of the invention, the magnetic field may be either a magnetostatic field or may have a constant component.

The methods according to the invention are realized by using a device comprising a controllable source of current pulses supplied to at least one inductor. Said device is characterized in that it comprises a reservoir for the fluid medium adapted for a human body, with the inductors being disposed on the surface of the reservoir in the regions of contact with the human body. According to this invention, the inductors are predominantly disposed in the regions most closely fitting the main human organs. Further, this device is characterized in that the inductors are mounted in the reservoir openings and are provided with the hermetically sealed covers on the interior side of the reservoir.

In the device according to this invention, the inductor covers are made in the form of electromagnetic membranes to produce an additional acoustic and vibration action.

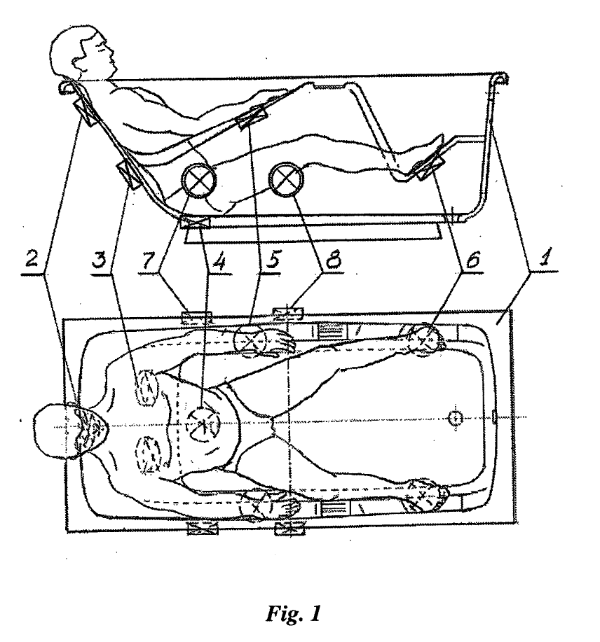

Realization of the methods and devices and embodiments are illustrated in FIGS. 1-4, wherein:

FIG. 1 illustrates an embodiment of the integrated hydro and magnetotherapy, with the magnets being disposed close to the human body organs.

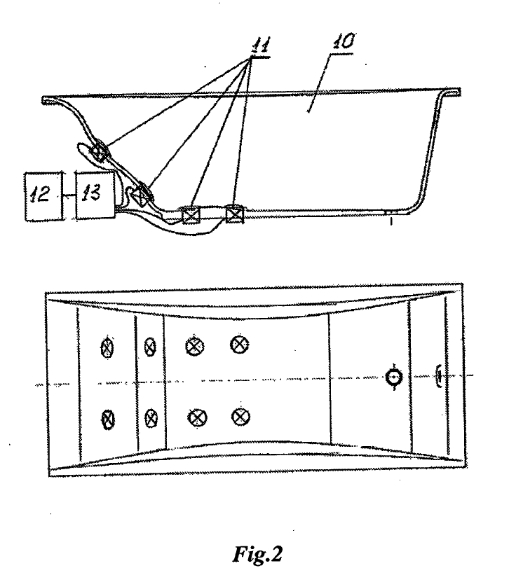

FIG. 2 illustrates an embodiment of the design of the bath and electronic devices for the integrated hydro and magnetotherapy.

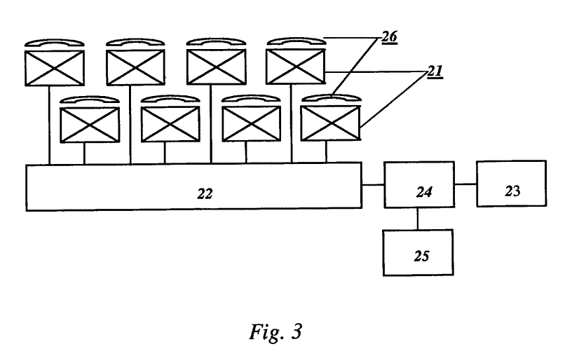

FIG. 3 illustrates an electric diagram of an embodiment of electronic and electromechanical devices for the integrated hydro-, acousto- and magnetotherapy and also for the hydromassage.

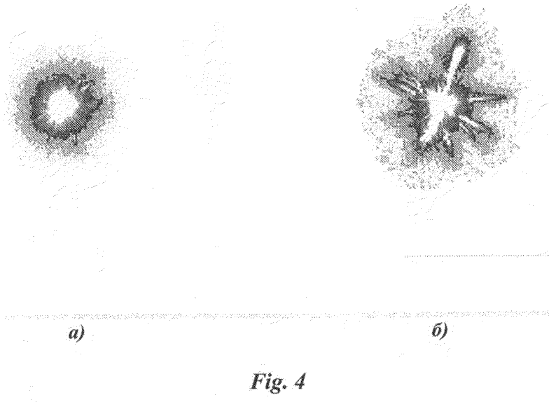

FIG. 4 shows images of a water drop obtained in the high-frequency fields prior and after the effect produced by the method according to this invention.

The embodiment illustrated in FIG. 1 is intended for preventing and treating diseases of the locomotor system and cardiovascular diseases.

The bath body 1 is adapted for a convenient and relaxed position and provided with hand supports and feet stops. The bath body may be made of acryl or other material being permeable for magnetic fields. Magnetic inductors are disposed on the exterior side of the bath in the regions of the closest contact of the human body organs to be treated. Such inductors may include as follows: a neck inductor 2; a lumbar inductor 3; a pelvic inductor 4; a carpal inductor 5; a feet inductor 6; hip inductors 7; and knee inductors 8. It should be noted that inductors 7 and 8 are not covered with a large body mass and may also serve for the total water treatment in the bath.

Positioning the inductors in such manner helps efficiently use them for treating and preventing diseases of the locomotor system, most important internal parts of the body located in the lumbar part of the body such as a liver, kidneys and a spleen. The total exposure also efficiently effects the cardio-vascular system by improving blood characteristics, reducing viscosity, increasing vessel permeability, etc.

In addition, the presence of water both increases efficiency of the effect by cleansing the skin surface, pores, intercellular spaces and by dissolving salts and other substances, and it also may produce a thermal effect due to the thermal exposure.

Medicinal and aromatic preparations may be also added to water.

The other embodiment of disposal of the inductors illustrated in FIG. 2 allows a dynamic action and a combined action to be applied.

Inductors 11 in the bath 10 are distributed in the region where the larger mass of the human body is located and are connected to a current pulse generator 12 through a modulating and controlling device 13.

The generator 12 generates current pulse packets with the pulse-repetition frequency in the range of 1-10 Hz and the pulse repetition frequency in the packet of about 100 Hz, with amplitude being sufficient to produce the magnetic field within the range of 10-50 mTl, preferably, 10 mTl. The modulating and controlling device 13 distributes pulses across the inductors according to the defined program and simultaneously may modulate their amplitude and other parameters according to the harmonic or musical component selected from a special signal or recording of any musical composition.

The electronic circuit diagram illustrated in FIG. 3 comprises inductors distributed across the bath and connected to the software switching device 22 the input of which is coupled to the current pulse generator 23 via a modulator 24. A modulating harmonic or musical signal is fed to the modulator control input from a playback device 25.

To simultaneously provide a mechanical and vibration action, magnetic membranes 26, which efficiently transmit acoustic and mechanical action to the fluid and human body are disposed over the inductors 21 on the interior side of the bath. The membranes are directly vibrated by the modulated magnetic field in synchronized manner therewith, thereby enhancing the effect, while the fluid medium serves as an efficient sound conveying medium.

The studies have demonstrated that an effective fluid structure changes when it is exposed to the magnetic field modulated in that manner. FIG. 4 illustrates an image of a water drop in high-frequency fields prior a) and after b) treatment with magnetic field pulses using the INTERSPOK™ device according to the invention. Surprisingly, but such a structure remained unchanged for 4 days, i.e. the medical effect in liquid structures of the human body continues for a lengthy period of time even after the procedures were completed according to the invention.

Clinical trials have demonstrated high efficiency of the method used and no complications have been detected.

Therefore, the proposed invention represents an integrated and effective hydromagnetotherapy realized by using the device of the invention and allowing medical results to be obtained without using the above means separately and resorting to surgical or biochemical operation, although accompanying medicaments may also be used subject to the physician's prescription. The proposed invention may also be used for medical and preventive purposes in clinical and domestic conditions. Taking into consideration the fact that magnetic pulses are generated by current pulses in the inductors and may be low-voltage pulses, the proposed invention is safe for use in combination with the hydrotherapy.

1. A method for hydromagnetotherapy and/or prophylaxis of human diseases comprising the action of a fluid medium at least on a part of a human body, characterized in that the part of the body immersed in the fluid medium is additionally exposed to the magnetic field.

2. The method for hydromagnetotherapy and/or prophylaxis of human diseases comprising the action of the fluid medium on at least a part of the human body and exposure of the fluid medium to the magnetic field, characterized in that the immersed part of the body is exposed to the pulsed magnetic field with the preset magnetic field pulse frequency and amplitude for a specific period of time.

3. The method for hydromagnetotherapy and/or prophylaxis of human diseases comprising the action of the fluid medium on at least the part of the human body with an additional acoustic exposure, characterized in that the immersed part of the body is additionally exposed to the pulsed magnetic field with the preset magnetic field pulse frequency and amplitude for a specific period of time.

4. The method for hydromagnetotherapy and/or prophylaxis of human diseases involving immersion of at least a part of the human body, preferably close to the organ to be treated, in the fluid medium, vibration action on the immersed part of the body, characterized in that a simultaneous exposure to sound and magnetic field is provided as a combined acoustic, vibration and magnetic field exposure.

5. The method according to claim 1, characterized in that the body is immersed in the fluid medium and the inductors are disposed along the body and in proximity to the organs to be treated.

6. The method according to claim 1, characterized in that the magnetic field is a magnetostatic field.

7. The method according to claim 5, characterized in that the organ to be treated is exposed to vibration action simultaneously with the acoustic and magnetic exposure, with the acoustic and vibration action being transmitted through the fluid medium.

8. The device for realizing the methods according to claim 1, comprising a reservoir for the fluid medium adapted for the human body, characterized in that the device comprises a controllable source of current pulses fed to at least one inductor, with the inductors being disposed on the surface of the reservoir in the regions of contact with the human body.

9. The device according to claim 8, characterized in that the inductors are predominantly disposed in the regions of the closest contact with the main human organs.

10. The device according to claim 8, characterized in that the inductors are mounted in the reservoir openings and are provided with the hermetically sealed covers on the interior side of the reservoir.

11. The device according to claim 10, characterized in that the covers are made in the form of electromagnetic membranes to produce an additional acoustic and vibration action.

12. The device according to claim 8, characterized in that the inductors are a distributed set of inductors, while the magnetic field is a traveling magnetic field.
